(19) United States
(12) Patent Application Publication
(10) Pub. No.: US 2020/0171253 A1
(43) Pub. Date: Jun. 4, 2020

(54) RESUSCITATION BAG WITH DERIVATION CONDUCT COMPATIBLE WITH THORACIC COMPRESSIONS

(71) Applicants: Air Liquide Santé (International), Paris (FR); Air Liquide Medical Systems, Antony (FR)

(72) Inventors: Thierry BOULANGER, Media, PA (US); Jean-Christophe RICHARD, Antony (FR); Marceau RIGOLLOT, Montrouge (FR); Jean-Marc GINER, Puteaux (FR)

(21) Appl. No.: 16/621,588

(22) PCT Filed: Nov. 30, 2017
(86) PCT No.: PCT/EP2017/080975

§ 371 Date: Dec. 11, 2019

Related U.S. Application Data
(60) Provisional application No. 62/525,421, filed on Jun. 27, 2017.

(51) Int. Cl. A61M 16/00; A61M 16/08; A61M 16/10; A61M 16/20; A61M 16/12

(52) U.S. Cl. CPC A61M 16/0078; A61M 16/0875; A61M 2202/0208; A61M 16/208; A61M 16/125; A61M 16/1005

(57) ABSTRACT

Artificial resuscitation bag (5) comprising a deformable bag (54) comprising a gas inlet (54a) and a gas outlet (54b), a gas conduit (47) in fluid communication with the gas outlet (54b) of the deformable bag (54), and a pneumatic valve (50) comprising an exhaust port (50c) cooperating with a membrane element (50b) for controlling the flow of gas exiting to the atmosphere through said exhaust port (50c), said membrane element (50b) being arranged into an inner compartment (50f) of the pneumatic valve (50).

### CROSS REFERENCE TO RELATED APPLICATION

This application claims the benefit of priority to U.S. Provisional Patent Application No. 62/525,421, filed Jun. 27, 2017, the entire contents of which are incorporated herein by reference.

### BACKGROUND

The present invention relates to an artificial respiration device, namely an artificial resuscitation bag that can be used for resuscitating a person, i.e. a patient, in state of cardiac arrest, and an installation comprising such an artificial resuscitation bag for resuscitating a person in state of cardiac arrest.

Cardiac arrest is a condition affecting hundreds of thousand people every year with a very poor prognosis.

One of the main lifesaving actions is to apply thoracic compressions or ‘TCs’ along with brief intervals of lung ventilation with a resuscitation bag. TCs are successive compressions and decompressions exerted on the thoracic cage of the person, i.e. the patient, in cardiac arrest. TCs aim at partially restoring inhalation and exhalation phases and therefore gas exchanges in the lungs, as well as promoting or restoring blood circulation toward the organs and especially the brain of the patient.

As these compressions and decompressions only mobilize small volumes of gas in and out of the patient's airways, it is advocated to perform regularly further gas insufflations to bring fresh O₂-containing gas into the lungs thereby enhancing the gas exchanges.

Usually, fresh O₂-containing gas is delivered by a resuscitation bag linked with an oxygen source and connected to the patient through a respiratory interface, typically a facial mask, a laryngeal mask, or an endotracheal tube.

To date, it is recommended to interpose 2 insufflations every 30 chest compressions, whereas the ideal rate of compressions, according to international guidelines, is between 100 and 120 compressions per minute (c/min).

However, several studies have shown that it is difficult for rescuers to correctly perform the resuscitation sequence and that the interruptions of TCs to initiate the insufflations with a resuscitation bag are often too long and deleterious with respect to the patient's outcome, as rapidly affecting the hemodynamic, i.e., in other words, offsetting the benefits of the TCs themselves.

A main goal of the present invention is to fix the problem encountered with current resuscitation bags, in particular to provide an improved resuscitation bag allowing continuous TCs and, when required, enabling insufflations of given volumes of fresh O₂-containing gas.

### SUMMARY

A solution according to the present invention concerns an artificial resuscitation bag comprising:
a deformable bag comprising a gas inlet and a gas outlet,
a gas conduit in fluid communication with the gas outlet of the deformable bag, and
a pneumatic valve comprising of an exhaust port cooperating with a membrane element for controlling the flow of gas exiting to the atmosphere through said exhaust port, said membrane element being arranged into an inner compartment of the pneumatic valve,

and further comprising:
an overpressure valve arranged in the gas conduit,
a first one-way valve arranged in the gas conduit between the overpressure valve and the pneumatic valve and
a derivation conduct having:
i) a first end fluidly connected to the gas conduit, between the gas outlet of the deformable bag and the overpressure valve, and
ii) a second end fluidly connected to the inner compartment of the pneumatic valve.

Depending on the embodiment, an artificial resuscitation bag according to the present invention can comprise of one or several of the following additional features:
the artificial resuscitation bag comprises a gas delivery conduit in fluid communication with the gas conduit for conveying at least part of the gas circulating into the gas conduit to a patient interface.
the patient interface comprises of a respiratory mask or a tracheal cannula.
the gas conduit conveys at least a part of the gas exiting the deformable bag through the gas outlet.
the overpressure valve is configured to vent to the atmosphere at least part of the gas present in the gas conduit, when the gas pressure in the gas conduit exceeds a given threshold-value.
the first one-way valve is configured for allowing a circulation of gas in the gas conduit only in the direction from the deformable bag toward the pneumatic valve.
the artificial resuscitation bag further comprises of a second one-way valve arranged in a conduit in fluid communication with the gas inlet of the deformable bag.
the pneumatic valve further comprises of a spring element acting on the membrane element for controlling the flow of gas exiting to the atmosphere through said exhaust port.
the spring element is arranged into the inner compartment of the pneumatic valve.
the pneumatic valve is arranged in the gas conduit.
the pneumatic valve is arranged in patient interface.
it further comprises a second one-way valve arranged in a first conduit in fluid communication with the gas inlet of the deformable bag.
the pneumatic valve further comprises a spring element acting on the membrane element for controlling the flow of gas exiting to the atmosphere through said exhaust port.
the spring element is arranged into the inner compartment of the pneumatic valve.
it further comprises a first conduit in fluid communication with the gas inlet of the deformable bag and an oxygen line fluidly connected to said first conduit.
it further comprises an oxygen distribution system comprising a gas distributor and a by-pass line connected to said gas distributor.
the gas distributor is arranged on the oxygen line.
the by-pass line is fluidly connected to the gas distributor and to the patient interface.

Further, the present invention also concerns an installation for resuscitating a person in state of cardiac arrest comprising:
an artificial resuscitation bag according to the present invention, and
an O₂ source fluidly connected to the artificial resuscitation bag by means of an oxygen line, for providing oxygen to said artificial resuscitation bag.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 represents an embodiment of the resuscitation bag according to the prior art.

FIG. 2 illustrates an embodiment of the resuscitation bag according to the prior art.

FIG. 3A illustrates an embodiment of the resuscitation bag according to the prior art.

FIG. 3B illustrates an embodiment of the resuscitation bag according to the prior art.

FIG. 4 illustrates an embodiment of the resuscitation bag according to the prior art.

FIG. 5 illustrates an embodiment of the resuscitation bag according to the prior art.

FIG. 6 illustrates an embodiment of the resuscitation bag according to the prior art.

FIG. 7A illustrates an embodiment of the resuscitation bag according to the present invention.

FIG. 7B illustrates an embodiment of the resuscitation bag according to the present invention.

FIG. 7C illustrates an embodiment of the resuscitation bag according to the present invention.

FIG. 8 illustrates an embodiment of the resuscitation bag according to the present invention.

FIG. 9 illustrates an embodiment of the resuscitation bag according to the present invention.

FIG. 10 illustrates an embodiment of the resuscitation bag according to the present invention.

FIG. 11 illustrates an embodiment of the resuscitation bag according to the present invention.

FIG. 12 is another embodiment of the resuscitation bag according to the present invention.

### DESCRIPTION OF PREFERRED EMBODIMENTS

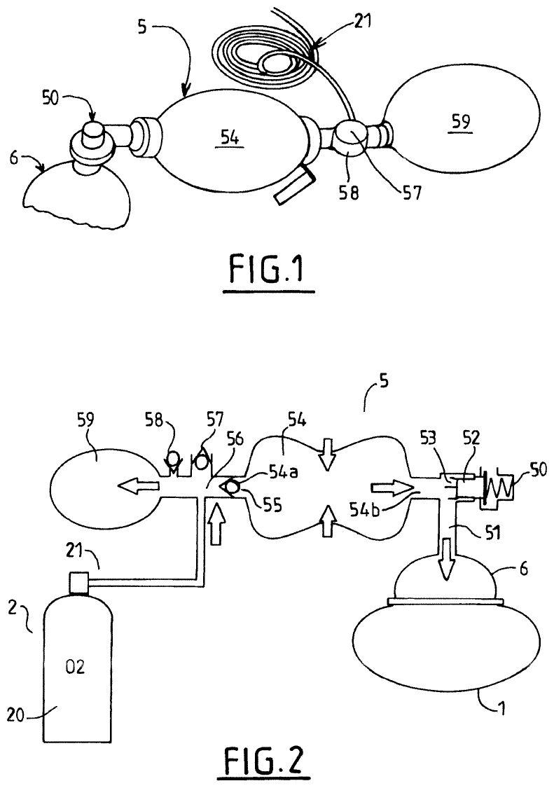

FIGS. 1 and 2 show a commercially available resuscitation bag 5 comprising of a respiratory interface 6 for feeding a respiratory gas to a patient, typically a respiratory mask, a flexible bag 54, and a valve element 50, such as a PEP valve, for diverting the gas in and out of the patient, during insufflation and exsufflation phases, and a source of an oxygen-containing gas 2, such as or including a gas cylinder 20 containing oxygen, which is delivered during insufflation phases.

The flexible bag 54 is filled with fresh gas formed by a mixture of oxygen provided by an oxygen line 21 connected to the oxygen source 2 (cf. FIG. 2), typically a medical grade oxygen cylinder 20, and ambient air provided by an admission valve 57 in fluid communication with the ambient atmosphere.

A supplementary gas reservoir 59 can be added to increase the availability of oxygen.

In FIG. 2, a patient 1 is connected to the resuscitation bag 5, via a respiratory interface 6, e.g. a facial mask, a laryngeal mask or similar.

The oxygen source 2, typically a cylinder 20 of medical grade oxygen, is fluidly connected via an oxygen line or tubing 21 and a first conduit element 56, to the flexible bag 54, the tubing 21 being fluidly connected to the first conduit element 56. The first conduit element 56 is further fluidly communicating with the inlet orifice 54a of the flexible bag 54. The first conduit element 56 is arranged between the supplementary gas reservoir 59 and the flexible bag 54.

Further, a first exhaust valve 58 is arranged in the first conduit element 56 for venting gas in the case of overpressure in the first conduit element 56.

When the operator squeezes the flexible bag 54 to perform an insufflation of gas to the patient 1, the flow of gas exiting the flexible bag 54 through its outlet orifice 54b travels to the patient 1 into the lumen of a second conduit 51 that is fluidly connected to the respiratory interface 6, such as a facial mask. At the same time, the flow of gas exiting the flexible bag 54 occludes the exhalation port 52 of a second exhaust valve 53 that is arranged in the second conduit 51, i.e. downstream of gas bag 54, as shown in FIG. 2.

This generates positive pressure which, as a result, forces a second one-way valve 55 arranged upstream of the bag 54 to close thereby preventing the gas of bag 54 to flow backward, i.e. in the first conduit 56, and to escape via the first exhaust valve 58. Meanwhile, the flow of oxygen travelling in tubing 21 enters into the first conduit 56 element and fills the supplementary reservoir 59 that is fluidly connected to first conduit element 56.

Due to the slightly positive pressure in first conduit element 56, the air admission valve 57 is closed. In the case where the reservoir 59 becomes over-distended by the entering flow of gas, a pressure increase will occur in first conduit element 56 and the gas in excess will be vented to the ambient atmosphere by the first exhaust valve 58. The opening pressure of the first exhaust valve 58 is close to 0, but slightly positive due to mechanical frictions.

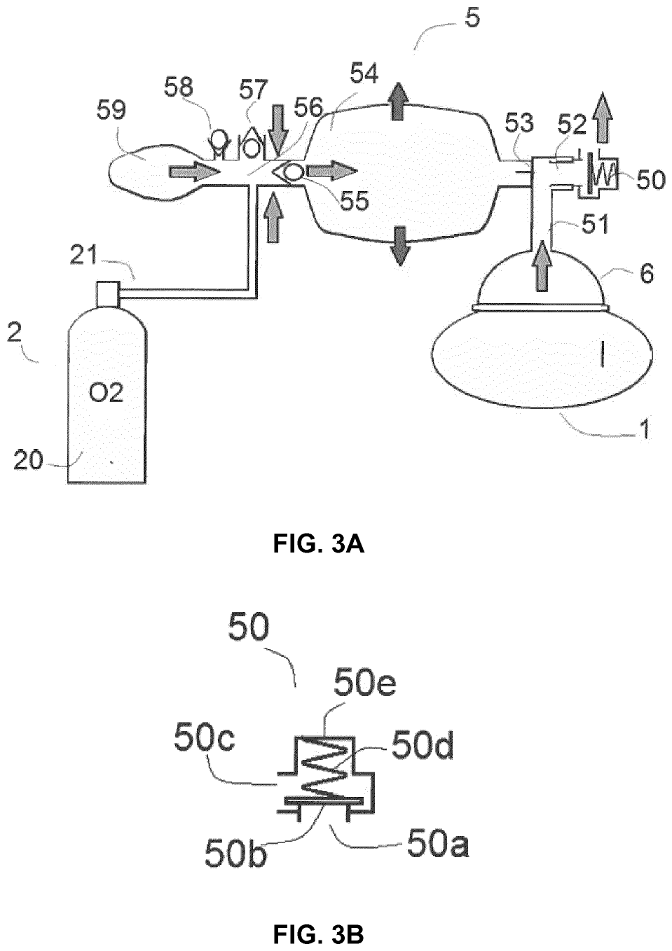

FIG. 3A shows an expiration phase of the commercially available resuscitation bag 5 of FIGS. 1 and 2, when the operator has stopped squeezing the bag 54, which bag 54 enters in an expansion phase due to a negative pressure that holds back the second exhaust valve 53 thereby opening the exhalation port 52. The volume of gas accumulated in the patient's airways during the preceding inspiratory phase will travel through interface 6 and second conduit 51 before being vented to ambient atmosphere through the exhalation port 52.

The resuscitation bag 5 includes a valve element 50 or PEP valve 50 (PEP=Positive Expiration Pressure) that creates a positive expiratory pressure, during exhalation phases, thereby helping keeping open the alveoli of the lungs of patient 1.

As detailed in FIG. 3B, such a PEP valve 50 typically comprises a spring 50d arranged in a housing 50e, which applies a constant force on a membrane 50b. The gas pressure in the PEP valve inlet port 50a, that is in fluid communication with exhalation port 52 and that applies on said membrane 50b, has to be sufficiently high for exerting a force greater than the load of the spring 50d for displacing the membrane 50b backward and opening a fluidic pathway between the inlet port 50a and an outlet port 50c of the PEP valve inlet port 50a. The fluidic pathway allows the gas pressure to escape through the outlet port 50c, thereby allowing an expiration of gas by the patient 1. It is possible to set the load of spring 50d to different expiratory pressures, such as expiratory pressures corresponding to 5 cm H₂O, 10 cm H₂O, or 20 cm H₂O.

At the same time, the negative pressure generated in bag 54 will open the second one-way valve 55 that will: i) direct the gas flow from tubing 21 into bag 54 via conduit 56, ii) empty reservoir 59 into bag 54 via conduit 56, and iii) open the air admission valve 57 thereby allowing ambient air entering successively into conduit 56 and bag 54, as shown in FIG. 3A.

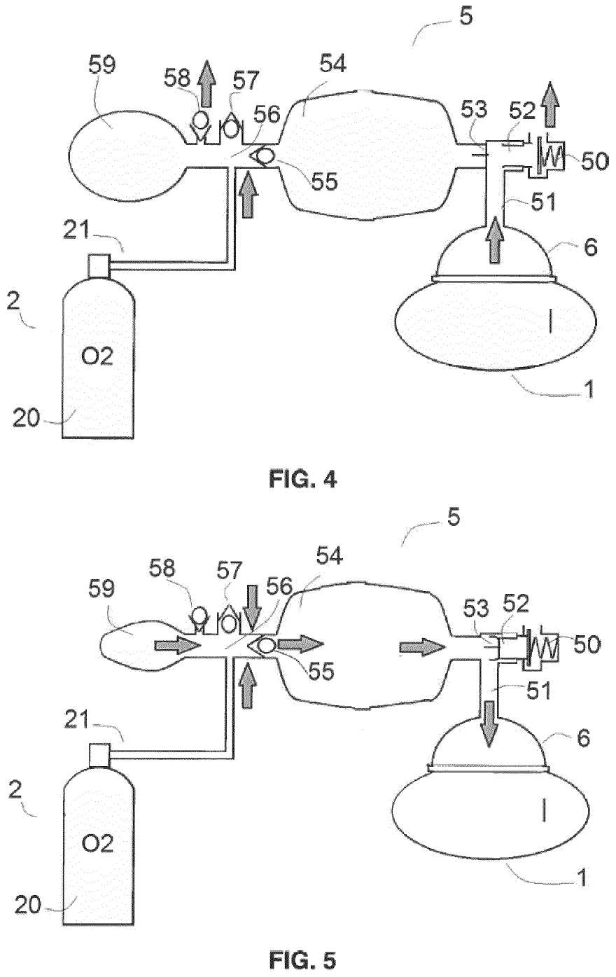

Further, FIGS. 4-6 show a sequence of thoracic compressions (TC) in association with the resuscitation bag 5 of FIGS. 1, 2 and 3A.

In FIG. 4, the resuscitation bag 5 is represented in its “rest” state, i.e. not active state, for example as it is before being used. The gas bag 54 and reservoir 59 are filled with gas and ready for an insufflation. The oxygen flowing from cylinder 20 and tubing 21 enters conduit 56 and is vented to the atmosphere through fist exhaust valve 58 acting as a safety valve.

When the bag 54 is in its “rest” state, the operator usually starts to exert thoracic compressions or TCs on the patient 1. Due to the TCs, the second exhaust valve 53 is pushed back, i.e. closed, thereby occluding the fluidic pathway 52 between gas bag 54 and second conduit 51. Indeed, a TC expels a small volume of gas from the patient's airways which travels backwardly through second conduit 51, exhaust port 52, and PEP valve 50. Actually, PEP valve 50 creates a resistance force against expired gases, which will promote or restore blood circulation in the patient's body.

When a TC is relaxed, the patient 1 enters decompression and the airway pressure becomes negative as shown in FIG. 5. The negative pressure closes PEP valve 50, i.e. occlude the fluidic passage between ports 50a and 50c (cf. FIG. 3B), and air is delivered by bag 54, thereby pushing the second exhaust valve 53 toward the exhaust port 52 for creating a fluidic passage between said gas bag 54 and second conduit 51.

Meanwhile, the second one-way valve 55 allows: i) a first flow of gas, e.g. oxygen, to travel in tubing 21 and first conduit element 56, and ii) a second flow of gas to exit reservoir 59 and to travel in first conduit element 56.

Further, a third flow of gas, i.e. air, is allowed to penetrate into conduit 56 via the admission valve 57, i.e. another one-way valve. These three flows of gas enter into bag 54 thereby filling said bag 54.

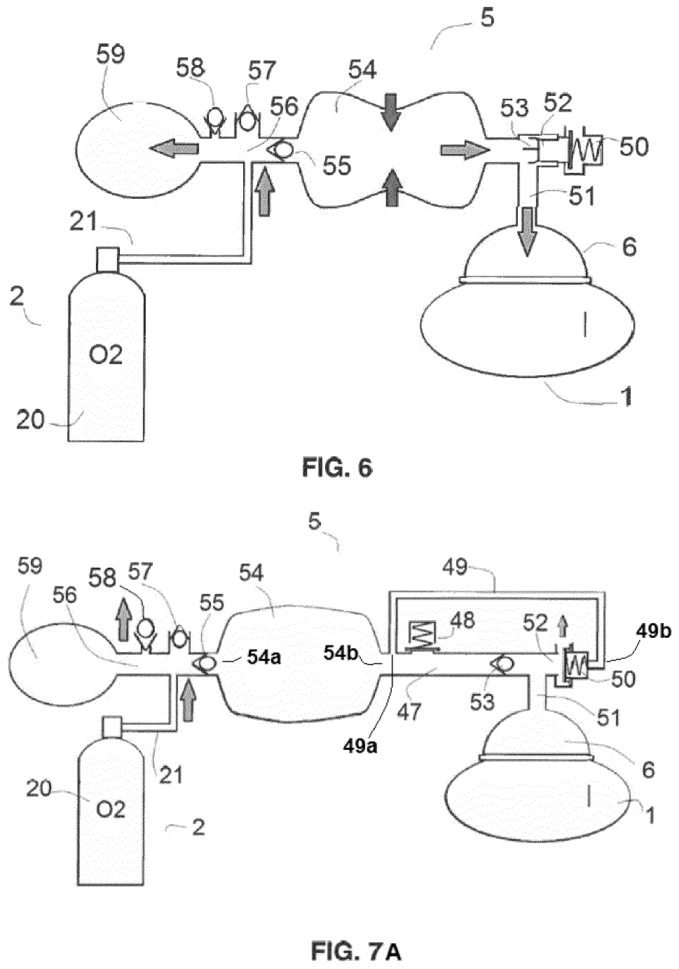

However, with such architecture a hazardous situation may exist as shown in FIG. 6: when the operator performs an insufflation as described earlier, if a TC occurs during this insufflation phase, exhaust valve 53 and second one-way valve 55 will prevent any gas exhaust. This constitutes a risk for the patient 1 as an over-pressure will appear, which can be deleterious for the lungs of the patient 1.

As shown in FIGS. 1-6, artificial resuscitation bags of the prior art do not allow to simultaneously performing safe and effective TCs and gas insufflations with the resuscitation bag. Indeed, with such known resuscitation bags, it is impossible to provide TCs during insufflations phases without risking over-pressures in the lungs which will negatively impact outcomes for the patient. This is the reason why TCs must be interrupted when an insufflation is required.

The present invention proposes an artificial resuscitation bag 5 that can overcome the above issue.

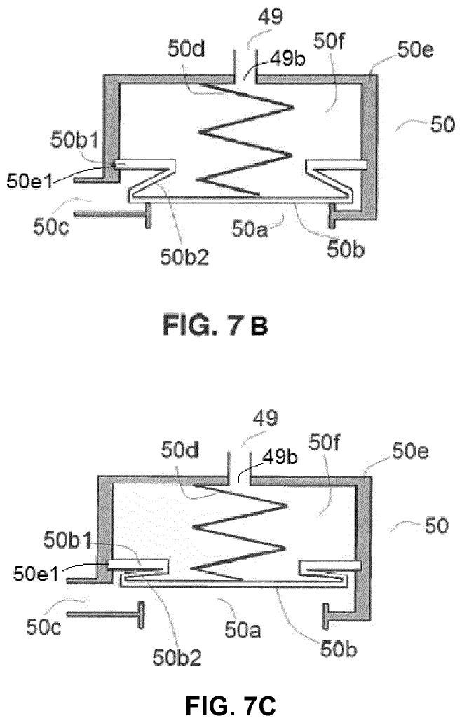

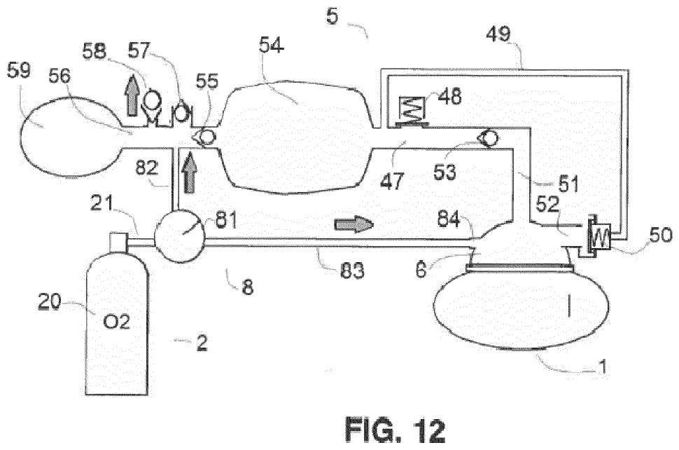

A first embodiment of an artificial resuscitation bag 5 according to the present invention is shown in FIGS. 7-11, whereas a second embodiment of an artificial resuscitation bag 5 according to the present invention is shown in FIG. 12.

FIG. 7A shows a first embodiment of a resuscitation bag 5 according to the present invention, allowing TCs to be performed while insufflating gas.

In FIG. 7A, the resuscitation bag 5 is in an initial state or “rest” state in case of a thoracic compression. The reservoir 59 is filled with oxygen, the oxygen being provided by the O₂ source 2, namely the cylinder 20 delivering oxygen to reservoir 59 via tubing 21 and first conduit 56. The first exhaust valve 58 is opened and vents the excess of oxygen to the atmosphere. The slight positive pressure in first conduit 56 keeps the one-way admission valve 57 closed. By mechanical transmission, this pressure will be equalized in all the parts behind the second one-way valve 55, i.e. into bag 54 and subsequent components, such as conduits 47, 51 and 52.

The control valve 50 of FIGS. 7B/7C works in differential mode. A deformable membrane 50b is tightly attached by its lips 50b1 to grooves 50e1 in a rigid structure 50e, which is the control valve 50 housing. A deformable portion 50b2 of membrane 50b helps this membrane 50b move forward or backward, depending on the conditions. At rest, this membrane 50b prevents a fluidic connection between the inlet conduit 50a and outlet conduit 50c, as shown in FIG. 7B. This is due to the force exerted by load spring 50d on the membrane 50b, as described previously. The force exerted by the load spring can be variable and set for example in a way that a pressure of 5 cm H₂O in inlet 50a is necessary to move the membrane 50b backward to perform a fluidic connection between inlet 50a and outlet 50c, as shown in FIG. 7C.

However, the control valve 50 of FIGS. 7B/7C has a chamber 50f which is fluidically connected to a derivation conduct 49 comprising a first end 49a fluidly connected to the gas conduit 47, between the gas outlet 54b of the deformable bag 54 and the overpressure valve 48, and a second end 49b fluidly connected to the inner compartment 50f of the pneumatic valve 50, as shown in FIG. 7A. Should the derivation conduct 49 provide a positive pressure, this pressure would add a force on top of the load spring 50d which will in turn make it harder to open the fluidic connection between inlet 50a and outlet 50c, unless the pressure at inlet 50a follows the increase of pressure in chamber 50f, offsetting its effect.

As shown in FIG. 7A, at the very onset of the TC, the pressure in conduits 47 and 51, in derivation conduct 49 and consequently in chamber 50f of the control valve 50 are equal. This means that only the load spring 50d will oppose the rise of pressure resulting from the TC. Following the example set above, as soon as the pressure will exceed 5 cm H₂O in second conduit 51, thus closing the first one-way valve 53, also called second exhalation valve 53, the control valve 50 will open to make a fluidic connection between inlet 50a and outlet 50c, allowing the volume expelled by the patient 1 to travel through interface 6, conduits 51 and 52, inlet 50a and exhaust port, or outlet 50c.

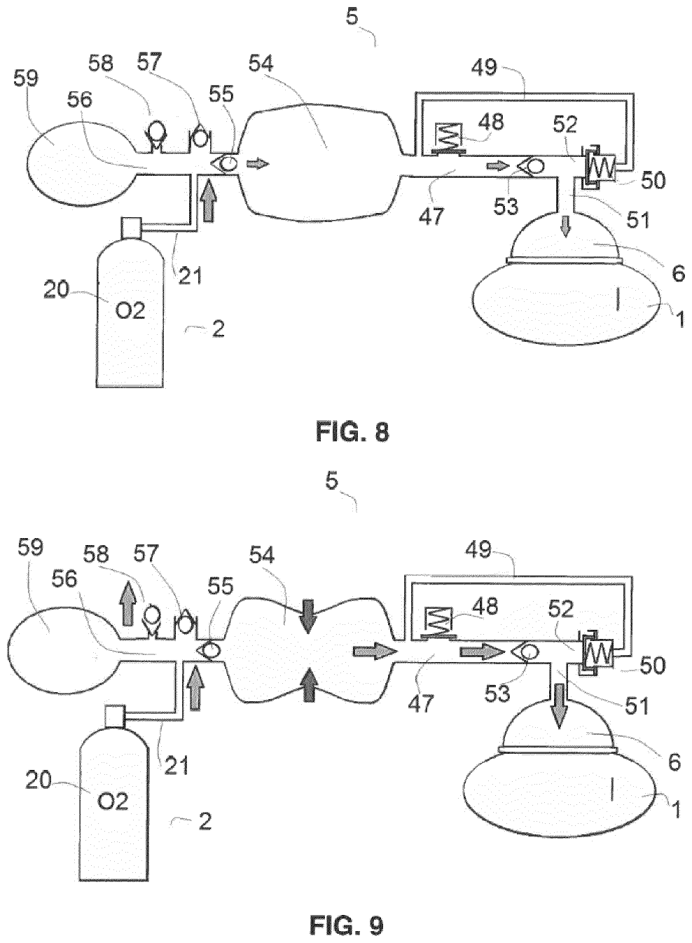

After the TC, follows a decompression phase as shown in FIG. 8. The pressure in the patient's airways suddenly decreases to potentially sub-atmospheric pressures. As a consequence, the flow of oxygen in first conduit 56, coming from tubing 21, will be directed to the patient 1 to offset this decrease in pressure, opening the second one-way valve 55 and the second exhalation valve 53, and traveling through bag 54 and conduits 47, 51 and interface 6. In addition, the pressure across the control valve 50, which is between derivation conduct 49 and therefore chamber 50f, and conduit 52 and therefore inlet 50a will be close to 0 and as a result the control valve 50 will be closed.

As a result, a direct fluidic pathway will be created between the oxygen supply in tubing 21 and patient 1.

In FIG. 9, the operator starts an insufflation by squeezing the bag 54 which will in turn open the first one-way valve 53. By the same mechanism, the pressure across the control valve 50, which is between derivation conduct 49 and therefore chamber 50f, and conduit 52 and therefore inlet 50a will be close to 0. As a result, the control valve 50 will remain closed, although the insufflation will create an increase in pressure in both sides of the control valve 50. As a consequence, all the gas exiting the bag 54 will travel into conduits 47 and 51 and be delivered to the patient 1 via interface 6.

On the other end of the bag, such positive pressure in the bag 54 will force the second one-way valve 55 to close and the oxygen coming from tubing 21 and entering conduit 56 will either fill the reservoir 59 or vent to the atmosphere through exhaust valve 58.

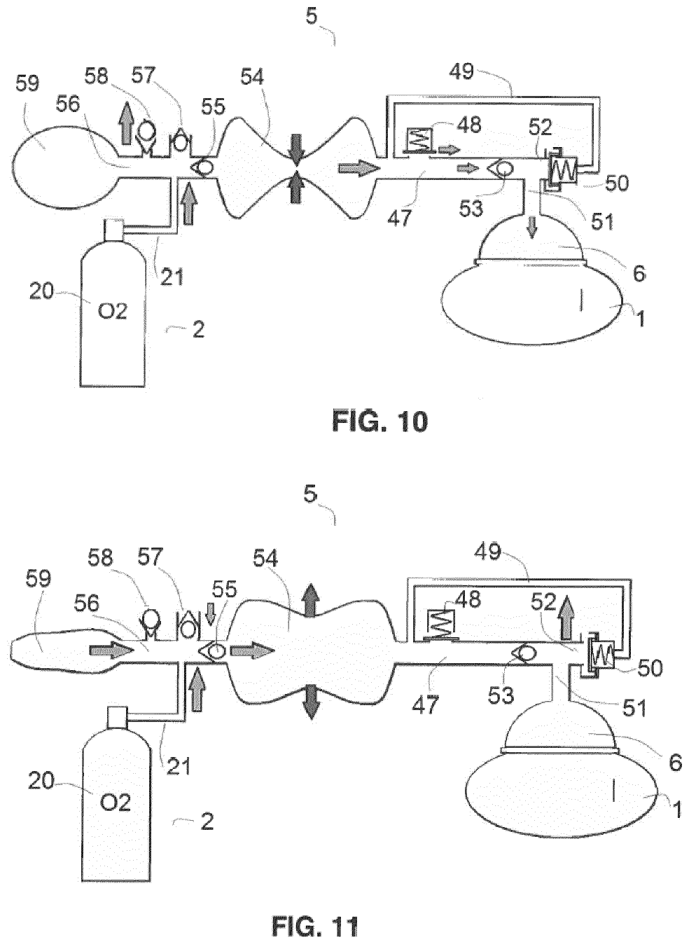

At some point during the insufflations, the pressure may become too high. The resuscitation bag of the present invention provides a means to control this pressure as shown in FIG. 10. This function is made possible by PPEAK valve 48 which is of the same construction as the PEP valve 50 hereabove described (cf. FIG. 3B), although its load spring is set in a way that only a pressure greater than 20 cm H₂O, for example, opens it and limits the pressure into conduits 47, 51 and patient's airways at this set value.

During the insufflation described with references to FIGS. 9 and 10, the control valve 50 assists the operator. Indeed, in case of a thoracic compression the pressure on the patient 1 side will increase, for instance above 20 cm H₂O if we consider the compression occurred while PPEAK valve 48 was limiting the pressure, and close the first one-way valve 53. This will create an imbalance in terms of pressure between conduits 51, 52 and inlet 50a and their counterpart, e.g. conduits 47, derivation conduct 49 and chamber 50f. As soon as this imbalance exceeds the spring load 50d, causing a differential pressure of 5 cm H₂O, the control valve 50 will open and make a fluidic connection between inlet 50a and outlet 50c, allowing the volume expelled by the patient 1 to travel through interface 6, conduits 51 and 52, inlet 50a and exhaust port, or outlet 50c.

FIG. 11 shows the expiration phase, when the operator has stopped squeezing the bag 54, which enters an expansion phase. This creates a negative pressure which will open the second one-way valve 55, which will in turn: i) direct flow from tubing 21 into bag 54 via the first conduit 56; ii) empty reservoir 59 into bag 54 via first conduit 56; and iii) open one-way admission valve 57 which will let ambient air flow into bag 54 via conduit 56.

The same negative pressure will hold back the first one-way valve 53, close PPEAK valve 48 and decrease the pressure in derivation conduct 49 which will in turn dramatically decrease the pressure in chamber 50f of control valve 50. As the pressure in the patient's airways is high as a consequence of the past insufflation, the control valve 50 opens to make a fluidic connection between inlet 50a and outlet 50c, allowing the volume expired by the patient 1 to travel through interface 6, conduits 51 and 52, inlet 50a and exhaust port, or outlet 50c. The control valve 50 will remain open until an equilibrium is met between pressure in conduits 51 and opening pressure of control valve 50, defined by spring load 50d which, by virtue of the description above, should be around. 5 cm H₂O. The patient 1 has returned to a low pressure level where subsequent thoracic compressions can occur, as described in FIG. 7A.

The resuscitation bag 5 of the present invention has the ability to allow safe insufflations by limiting the pressure at the patient's airways while authorizing compression phases, therefore optimizing hemodynamic of the patient.

The resuscitation bag 5 of FIGS. 7-11 constitutes a great improvement over those of the prior art.

A second embodiment of the resuscitation bag 5 according to the present invention that further enhances TCs, is shown in FIG. 12.

Following a TC, as shown in FIG. 7, the gas flowing into the patient 1, during the thoracic decompression phase as illustrated in FIG. 8, will partly be composed of the gas expelled from the patient 1 during TC and present in the interface 6 and conduits 51 and 52.

This gas contains a “high” level of CO₂, which replaces valuable oxygen and further prevents the CO₂ clearance from the lung.

In many cases, it is advantageous:
to lower as much as possible the space in which the CO₂ can be present, e.g. interface 6 and conduits 51 and 52, and
to “flush” out a maximum of CO₂ rich-gases, over the course of the resuscitation process.

In this aim, according to the second embodiment shown in FIG. 12, the control valve 50 is arranged directly in the region of the interface 6 so as to be fluidly connected to interface 6 via conduit 52. Thus, control valve 50 can more efficiently vent CO₂-enriched gases exhaled by patient 1 to the atmosphere, thereby avoiding CO₂ build-up into conduit 51. Further, an oxygen distribution system 8 comprising a by-pass line 83 and a gas distributor 81 is provided. The by-pass line 83 is arranged between the gas distributor 81 fed by the oxygen source 2 and the interface 6.

The inlet of the gas distributor 81 is fluidly connected to the oxygen source 2 via oxygen line or tubing 21. In other words, the gas distributor 81 is arranged on the oxygen line 21 as shown in FIG. 12.

The distributor 81, when manually operated by the operator, diverts a portion of the total incoming oxygen flow either to the downstream portion 82 of the oxygen line 21 that is connected to resuscitation bag 5 via the first conduit 56, or to the by-pass line 83 that is fluidly connected to the interface 6 via an admission port 84.

By acting on gas distributor 81, e.g. a proportional diverting valve, the operator can select/allocate the respective amounts of oxygen flowing into by-pass tubing 83 and further into the downstream portion 82 of the oxygen line 21. The first oxygen flow conveyed by the downstream portion 82 of the oxygen line 21 enters into the first conduit 56 and, as already explained (cf. FIGS. 7 and 8), when no gas insufflation is performed, helps keep a minimal positive pressure in the bag 54 and subsequent conduit 47, derivation conduct 49 and chamber 50f, thanks to exhaust valve 58.

Further, the second oxygen flow conveyed by the by-pass tubing 83 enters into interface 6, such as a respiratory mask, via the admission port 84. As the oxygen flow is continuous, a pressure build-up occurs in interface 6 and conduit 52 and further a pressure imbalance across control valve 50 makes the fluidic connection between inlet conduit 50a and outlet conduit 50c to vent to the atmosphere, excessive flow, as hereinabove described in connection with FIGS. 7 to 11. Such a gas venting will also drag to the atmosphere any residual CO₂ from interface 6 and conduit 52. Vented CO₂ is substituted by fresh oxygen delivered by by-pass line 83.

Actually, an improved resuscitation bag according to the present invention brings the benefits to limit the level of CO₂ during the resuscitation process and promote oxygenation of the lungs.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited

1. An artificial resuscitation bag (5) comprising:
a deformable bag (54) comprising a gas inlet (54a) and a gas outlet (54b),
a gas conduit (47) in fluid communication with the gas outlet (54b) of the deformable bag (54), and
a pneumatic valve (50) comprising an exhaust port (50c) cooperating with a membrane element (50b) for controlling the flow of gas exiting to the atmosphere through said exhaust port (50c), said membrane element (50b) being arranged in an inner compartment (50f) of the pneumatic valve (50),
and further comprising:
an overpressure valve (48) arranged in the gas conduit (47),
a first one-way valve (53) arranged in the gas conduit (47) between the overpressure valve (48) and the pneumatic valve (50) and
a derivation conduct (49) having:
i) a first end (49a) fluidly connected to the gas conduit (47), between the gas outlet (54b) of the deformable bag (54) and the overpressure valve (48), and
ii) a second end (49b) fluidly connected to the inner compartment (50f) of the pneumatic valve (50).

2. The artificial resuscitation bag according to claim 1, further comprising a gas delivery conduit (51) in fluid communication with the gas conduit (47) configured and adapted for conveying at least part of the gas circulating into the gas conduit (47) to a patient interface (6).

3. The artificial resuscitation bag according to claim 1, wherein the gas conduit (47) is configured and adapted to convey at least a part of the gas exiting the deformable bag (54) through the gas outlet (54b).

4. The artificial resuscitation bag according to claim 1, wherein the overpressure valve (48) is configured and adapted to vent to the atmosphere at least part of the gas present in the gas conduit (47), when the gas pressure in the gas conduit (47) exceeds a given threshold-value.

5. The artificial resuscitation bag according to claim 1, wherein the first one-way valve (53) is configured and adapted for allowing a circulation of gas in the gas conduit (47) only in the direction from the deformable bag (54) toward the pneumatic valve (50).

6. The artificial resuscitation bag according to claim 1, wherein the pneumatic valve (50) is arranged in the gas conduit (47).

7. The artificial resuscitation bag according to claim 1, wherein the pneumatic valve (50) is arranged in patient interface (6).

8. The artificial resuscitation bag according to claim 1, further comprising a second one-way valve (55) arranged in a first conduit (56) in fluid communication with the gas inlet (54a) of the deformable bag (54).

9. The artificial resuscitation bag according to claim 1, wherein the pneumatic valve (50) further comprises a spring element (50d) acting on the membrane element (50b) for controlling the flow of gas exiting to the atmosphere through said exhaust port (50c).

10. The artificial resuscitation bag according to claim 9, wherein the spring element (50d) is arranged into the inner compartment (50f) of the pneumatic valve (50).

11. The artificial resuscitation bag according to claim 1, further comprising a first conduit (56) in fluid communication with the gas inlet (54a) of the deformable bag (54) and an oxygen line (21) fluidly connected to said first conduit (56).

12. The artificial resuscitation bag according to claim 1, further comprising an oxygen distribution system (8) comprising a gas distributor (81) and a by-pass line (83) connected to said gas distributor (81).

13. The artificial resuscitation bag according to claim 12, wherein the gas distributor (81) is arranged on the oxygen line (21).

14. The artificial resuscitation bag according to claim 12, wherein the by-pass line (83) is fluidly connected to the gas distributor (81) and to the patient interface (6).

15. An installation for resuscitating a person in state of cardiac arrest comprising:
an artificial resuscitation bag (5) according to claim 1, and
an O₂ source (2) fluidly connected to the artificial resuscitation bag (5) by an oxygen line (21), configured and adapted for providing oxygen to said artificial resuscitation bag (5).
